SEQUENCE LISTING (4 sequences)

SEQ ID NO: 1 - DNA, 22 bp, synthetic construct
  source 1..22: mol_type = other DNA
  misc_feature 1..22: note = Forward primer IL-1B-F for IL-1B gene
gcaactgttcctgaactcaact

SEQ ID NO: 2 - DNA, 21 bp, synthetic construct
  source 1..21: mol_type = other DNA
  misc_feature 1..21: note = Reverse primer IL-1B-R for IL-1B gene
atcttttggggtccgtcaact

SEQ ID NO: 3 - DNA, 24 bp, synthetic construct
  source 1..24: mol_type = other DNA
  misc_feature 1..24: note = Forward primer GAPDH-F for GAPDH gene
tggtatcgtggaaggactcatgac

SEQ ID NO: 4 - DNA, 24 bp, synthetic construct
  source 1..24: mol_type = other DNA
  misc_feature 1..24: note = Reverse primer GAPDH-R for GAPDH gene
atgccagtgagcttcccgttcagc

(19) United States
(12) Patent Application Publication
(10) Pub. No.: US 2024/0216286 A1
(43) Pub. Date: Jul. 4, 2024

(54) NANOSPHERE AND USE THEREOF

(71) Applicant: CHANG GUNG UNIVERSITY, Taoyuan City (TW)

(72) Inventors: Jui-Yang LAI, Taoyuan City (TW); Chia-Jung YANG, Taoyuan City (TW)

(21) Appl. No.: 18/204,780

(22) Filed: Jun. 1, 2023

(30) Foreign Application Priority Data
Dec. 30, 2022 (TW) 111150893

(51) Int. Cl. A61K 9/51; A61K 9/00; A61K 31/221; A61K 31/4439; A61K 31/53; A61K 31/573; A61P 27/02

(52) U.S. Cl. CPC A61K 9/5115; A61K 9/0048; A61K 9/5169; A61K 31/221; A61K 31/4439; A61K 31/53; A61K 31/573; A61P 27/02

(57) ABSTRACT

A nanosphere includes a metal oxide hollow nanosphere and a poly-L-histidine. The metal oxide is selected from the group consisting of cerium(IV) oxide (CeO2), aluminum oxide (Al2O3), copper(II) oxide (CuO), titanium dioxide (TiO2), sodium oxide (Na2O), zinc oxide (ZnO), gold(II) oxide (AuO), iron(II, III) oxide (Fe3O4), and combinations thereof. The poly-L-histidine is grafted on a surface of the metal oxide hollow nanosphere. A drug delivery carrier including the nanosphere, and a method for alleviating an anterior segment eye disease using the drug delivery carrier are also disclosed.

### CROSS-REFERENCE TO RELATED APPLICATION

This application claims priority to Taiwanese Invention Patent Application No. 111150893, filed on Dec. 30, 2022.

### REFERENCE TO SEQUENCE LISTING, TABLE OR COMPUTER PROGRAM

The Sequence Listing submitted concurrently herewith with a file name of “NP-35538.xml,” a creation date of Jan. 4, 2023, and a size of 5.57 kilobytes, is part of the specification and is incorporated by reference herein.

### FIELD

The disclosure relates to a nanosphere and a drug delivery carrier including the same. The disclosure also relates a method for alleviating an anterior segment eye disease using the drug delivery carrier.

### BACKGROUND

When an ocular surface comes into contact with chemicals, hot substances or pathogens, part of the anterior segment eye tissues, which includes cornea, iris, ciliary body, crystalline lens, may be injured, probably leading to an anterior segment eye disease, such as keratitis, uveitis, conjunctivitis, and endophthalmitis. The aforementioned anterior segment eye diseases, if not timely treated, may further lead to cataract, retinal damage or even blindness.

In clinic, a non-invasive treatment method, such as topical instillation, is often adopted for curing an anterior segment eye disease. Although ocular barriers (e.g., cornea impermeability, tear dilution, and lacrimal drainage) and corneal reflex (also known as blink reflex) can prevent microorganisms and impurities from entering into the eye chambers, they may hinder the penetration of ophthalmic drugs, causing difficulty in delivering the ophthalmic drugs to the anterior eye segment tissues, thus deceasing bioavailability and treatment effect of the ophthalmic drugs.

TW 1764564 B discloses a dual-functionalized ceria hollow nanosphere that is grafted with 4-(2-(7-amino-2-(furan-2-yl)-[1,2,4]triazolo[1,5-a][1,3,5]triazin-5-ylamino)ethyl)phenol (ZM-241385) and chitosan, and that can deliver an ophthalmic drug into the interior of an eye.

In spite of the aforesaid, there is still a need for those skilled in the art to develop a drug delivery carrier which can not only send an ophthalmic drug into the interior of an eye, but also accurately drop off and release the ophthalmic drug at the anterior eye segment tissues, so as to exert the therapeutic effect of the ophthalmic drug.

### SUMMARY

Accordingly, in a first aspect, the present disclosure provides a nanosphere, which can alleviate at least one of the drawbacks of the prior art.

The nanosphere includes: a metal oxide hollow nanosphere, the metal oxide being selected from the group consisting of cerium(IV) oxide (CeO₂), aluminum oxide (Al₂O₃), copper(II) oxide (CuO), titanium dioxide (TiO₂), sodium oxide (Na₂O), zinc oxide (ZnO), gold(II) oxide (AuO), iron(II, III) oxide (Fe₃O₄), and combinations thereof; and
a poly-L-histidine grafted on a surface of the metal oxide hollow nanosphere.

In a second aspect, the present disclosure provides a drug delivery carrier, which can alleviate at least one of the drawbacks of the prior art. The drug delivery carrier includes:
the aforesaid nanosphere; and
an ophthalmic drug.

In a third aspect, the present disclosure provides a method for alleviating an anterior segment eye disease, which can alleviate at least one of the drawbacks of the prior art. The method includes administrating to a subject in need thereof a pharmaceutical composition containing the aforesaid drug delivery carrier.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the Fourier Transform Infrared Spectroscopy (FTIR) spectra of ceria hollow nanospheres (CeO₂-HNS), poly-L-histidine functionalized ceria hollow nanospheres 1 to 3 (His-CeO₂-HNS1 to His-CeO₂-HNS3), and poly-L-histidine of Example 2, infra.

FIG. 2 shows the zeta (ξ) potentials of CeO₂-HNS, and His-CeO₂-HNS1 to His-CeO₂-HNS3 of Example 2, infra, in which the symbol represents p<0.05 (compared with CeO₂-HNS).

FIG. 3 shows the transmission electron microscope images of CeO₂-HNS, poly-L-histidine functionalized ceria nanoparticles (His-CeO₂-NP), and His-CeO₂-HNS1 to His-CeO₂-HNS3 of Example 2, infra.

FIG. 4 shows the permeability coefficient of the rabbit corneal epithelial cells (RCECs) determined in each group of Example 2, infra, in which the symbol “*” represents p<0.05 (compared with the normal control group).

FIG. 5 shows the encapsulation percentages of acetylcholine chloride (Ach) determined in CeO₂-HNS, and His-CeO₂-HNS1 to His-CeO₂-HNS3 of Example 3, infra, in which the symbol “*” represents p<0.05 (compared with CeO₂-HNS).

FIG. 6 shows the encapsulation percentages of 4-[4-(1,3-benzodioxol-5-yl)-5-(2-pyridinyl)-1H-imidazol-2-yl]benzamide (SB-431542) determined in CeO₂-HNS, and His-CeO₂-HNS1 to His-CeO₂-HNS3 of Example 3, infra, in which the symbol “*” represents p<0.05 (compared with CeO₂-HNS).

FIG. 7 shows the encapsulation ratios (%) of Ach and SB-431542 in CeO₂-HNS, and His-CeO₂-HNS1 to His-CeO₂-HNS3 of Example 3, infra.

FIG. 8 shows the relative content of cerium (Ce) in corneal tissues of the rats in each group of Example 4, infra.

FIG. 9 shows the evaluation of corneal haziness in each group of Example 5, infra.

FIG. 10 shows the histological observation of the corneal tissue in each group of Example 5, infra.

FIG. 11 shows the Young's modulus (MPa) determined in each group of Example 5, infra, in which the symbol “*” represents p<0.05 (compared with the pathological control group).

FIG. 12 shows the relative mRNA expression level of IL-1β gene determined in each group of Example 6, infra, in which the symbol represents p<0.05 (compared with the pathological control group).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

By conducting research, the applicants found that a ceria hollow nanosphere having a surface grafted with poly-L-histidine exhibits excellent cornea permeability, and hence can be used to deliver an ophthalmic drug enclosed therein to the anterior segment eye tissue, thereby rapidly releasing the ophthalmic drug.

Accordingly, the present disclosure provides a nanosphere, which includes:
a metal oxide hollow nanosphere, the metal oxide being selected from the group consisting of cerium(IV) oxide (CeO₂), aluminum oxide (Al₂O₃), copper(II) oxide (CuO), titanium dioxide (TiO₂), sodium oxide (Na₂O), zinc oxide (ZnO), gold(II) oxide (AuO), iron(II, III) oxide (Fe₃O₄), and combinations thereof; and
a poly-L-histidine grafted on a surface of the metal oxide hollow nanosphere.

In certain embodiments, the metal oxide is CeO₂.

According to the present disclosure, the metal oxide hollow nanosphere may have a particle size ranging from 20 nm to 150 nm. In certain embodiments, the metal oxide hollow nanosphere may have a particle size ranging from 40 nm to 100 nm. In an exemplary embodiment, the metal oxide hollow nanosphere is a CeO₂ hollow nanosphere having a particle size of 75 nm.

According to the present disclosure, the procedures and conditions for preparing the CeO₂ hollow nanosphere may be adjusted according to practical requirements. In this regard, those skilled in the art may refer to journal articles, e.g., Strandwitz N. C. and Stucky G. D. (2009), Chem. Master., 19:4577-4582.

According to the present disclosure, examples of a template suitable for preparing the CeO₂ hollow nanosphere may include, but are not limited to, tetraethyl orthosilicate (TEOS), glucose, and sucrose. In an exemplary embodiment, the template is TEOS.

According to the present disclosure, the poly-L-histidine suitable for use in this disclosure may be obtained as commercial products, or may be prepared using polymer synthesis techniques well-known to those skilled in the art.

According to the present disclosure, the nanosphere may be produced by subjecting the metal oxide hollow nanosphere and the poly-L-histidine to a grafting reaction, and a weight ratio of the metal oxide hollow nanosphere to the poly-L-histidine ranges from 1:0.25 to 1:1. In an exemplary embodiment, the weight ratio of the metal oxide hollow nanosphere to the poly-L-histidine is 1:1.

According to the present disclosure, the procedures and conditions for carrying out the grafting reaction may be adjusted according to practical requirements. In this regard, those skilled in the art may refer to journal article, e.g., Ostadhossein F. et al. (2018), Bioconjug. Chem., 29:3913-3922.

In an exemplary embodiment, the surface of the metal oxide hollow nanosphere is subjected to polyethylene glycol (PEG) modification before the grafting reaction.

According to the present disclosure, the procedures and conditions of the grafting reaction are within the expertise and routine skills of those skilled in the art.

According to the present disclosure, the grafting reaction may be carried out at a temperature ranging from 16° C. to 26° C. for a time period ranging from 18 hours to 24 hours. In an exemplary embodiment, the grafting reaction is carried out at 25° C. for 24 hours.

The present disclosure provides a drug delivery carrier, which includes the aforesaid nanosphere and an ophthalmic drug.

According to the present disclosure, a weight ratio of the nanosphere to the ophthalmic drug may range from 1:0.5 to 1:10. In an exemplary embodiment, the weight ratio of the nanosphere to the ophthalmic drug is 1:0.6. In another exemplary embodiment, the weight ratio of the nanosphere to the ophthalmic drug is 1:1.

According to the present disclosure, the ophthalmic drug may be selected from the group consisting of acetylcholine chloride (Ach), 4-[4-(1,3-benzodioxol-5-yl)-5-(2-pyridinyl)-1H-imidazol-2-yl]benzamide (SB-431542), tobramycin, penicillin, tetracycline, dexamethasone, hydrocortisone, hydrocortisone acetate, amcinonide, betamethasone, halometasone, clobetasone-17-butyrate, ascorbic acid, hyaluronic acid, and combinations thereof. In certain embodiments, the ophthalmic drug may be selected from the group consisting of Ach, SB-431542, dexamethasone, and combinations thereof. In an exemplary embodiment, the ophthalmic drug is a combination of Ach and SB-431542.

Since the drug delivery carrier according the present disclosure has been verified, by animal experiments, to effectively relieve structural and inflammatory damage of the eyes of rats which occurs as a result of corneal alkali burn, hence, the drug delivery carrier can be expected to be utilized in alleviating an anterior segment eye disease.

Therefore, the present disclosure provides a method for alleviating the aforesaid anterior segment eye disease, which includes administrating to a subject in need thereof a pharmaceutical composition containing the aforesaid drug delivery carrier.

According to the present disclosure, the anterior segment eye disease may be selected form the group consisting of ocular burn (e.g., ocular chemical burn and ocular thermal burn), keratitis (e.g., bacterial keratitis), uveitis, conjunctivitis, xerophthalmia, endophthalmitis, obstruction of meibomian glands, and combinations thereof. In certain embodiments, the anterior segment eye disease may be the ocular chemical burn selected from the group consisting of alkali burn and acid burn.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the pharmaceutical composition may be formulated into a dosage form suitable for intraocular administration or topical ophthalmic administration using technology well known to those skilled in the art.

According to the present disclosure, the dosage form suitable for topical ophthalmic administration includes, but is not limited to, drops, emulsions, gels, ointments, creams, sprays, micelles, and suspensions.

According to the present disclosure, the dosage form suitable for intraocular administration includes, but is not limited to, an injection, e.g., a sterile aqueous solution, a dispersion or an emulsion.

The pharmaceutical composition according to the present disclosure may be administered via one of the following routes: subtenon injection, intravitreal injection, intracameral injection, intra-retinal injection, subretinal injection, and suprachoroidal injection.

According to the present disclosure, the pharmaceutical composition may further include a pharmaceutically acceptable carrier widely employed in the art of drug-manufacturing. For instance, the pharmaceutically acceptable carrier may include one or more of the following agents: solvents (e.g., a sterile water), buffers (e.g., an ophthalmic balanced salt solution, phosphate buffered saline (PBS), Ringer's solution and Hank's solution), emulsifiers, suspending agents, decomposers, pH adjusting agents, stabilizing agents, chelating agents, preservatives, diluents, absorption delaying agents, liposomes, lubricants, and the like. The choice and amount of the aforesaid agents are within the expertise and routine skills of those skilled in the art.

The dose and frequency of administration of the pharmaceutical composition of the present disclosure may vary depending on the following factors: the severity of the illness or disorder to be treated, routes of administration, and age, physical condition and response of the subject to be treated. In general, the pharmaceutical composition may be administered in a single dose or in several doses.

The present disclosure will be further described by way of the following examples. However, it should be understood that the following examples are intended solely for the purpose of illustration and should not be construed as limiting the present disclosure in practice.

### Examples

### General Experimental Materials:

1. Cerium nitrate, tetraethyl orthosilicate (TEOS), ethanol, ethylene glycol, ammonium hydroxide (28% ammonia (NHs) in water), sodium hydroxide, [1-ethyl-3-(3-dimethylaminopropyl) carbodiimide hydrochloride] (EDC), poly-L-histidine, ninhydrin, lipopolysaccharide (LPS), acetylcholine chloride (Ach), 4-[4-(1,3-benzodioxol-5-yl)-5-(2-pyridinyl)-1H-imidazol-2-yl]benzamide (SB-431542; an inhibitor of an activin receptor-like kinase (ALK) receptor), and 4-(2-{[7-Amino-2-(furan-2-yl)[1,2,4]tiazolo[1,5-a][1,3,5]trazin-5-yl]amino}ethyl)phenol (ZM-241385; an adenosine A2A receptor antagonist) used in the following experiments were purchased from Sigma-Aldrich, St. Louis, Mo., USA.
2. Phosphonate-polyethylene glycol (PEG)-COOH (PO-PEG-COOH; molecular weight (Mw)=1200 g mol⁻¹; ref. SP-1P-10-001) used in the following experiments was purchased from Specific Polymers, Castries, France.
3. Chitosan (Cat. No. 28191) used in the following experiments was purchased from Fluka, Milwaukee, Wis., USA.
4. Experimental rats:

Male Sprague Dawley (SD) rats (5 to 8 weeks old, with a body weight of approximately 150 g to 250 g) used in the following experiments were purchased from BioLASCO Taiwan Co., Ltd. All the experimental rats were housed in an animal room with an independent air conditioning system under the following laboratory conditions: an alternating 12-hour light and 12-hour dark cycle, a temperature maintained at a range of 20° C. to 24° C., a relative humidity maintained at a range of 55% to 65%. Furthermore, water and food were provide ad libitum for all the experimental rats. All experimental procedures involving the experimental rats were in compliance with the guidelines of the Institutional Animal Care and Use Committee of Chang Gung Memorial Hospital and the Association for Research in Vision and Ophthalmology.
5. Cultivation and isolation of rabbit corneal epithelial cells (RCECs) and rabbit corneal keratocytes (RCKs)

Each of the RCECs and RCKs used in the following experiments were isolated from the corneal tissues of New Zealand white rabbits with reference to the method described in Lai J. Y. et al. (2012), Int. J. Nanomed., 7:1101-1114. The RCECs and RCKs were respectively grown in a 10 cm Petri dish containing Dulbecco's Modified Eagle's Medium (DMEM)/F12 (1:1 in volume ratio) (Gibco, Cat. No. 11330032) supplemented with 10% of fetal bovine serum (FBS), 10000 U/mL of penicillin, 10 mg/mL of streptomycin, and 25 μg/mL of amphotericin B. The RCECs and RCKs were cultivated in an incubator with culture conditions set at 37° C. and 5% CO₂.

### General Procedures:

### 1. Statistical Analysis:

The experimental data of all the test groups are expressed as mean±deviation (SD), and were analyzed using one-way analysis of variance (one-way ANOVA) followed by Tukey's test, so as to evaluate the differences between the groups. Statistical significance is indicated by p<0.05.

### Example 1. Preparation of Ceria Hollow Nanospheres

### A. Synthesis of Ceria Hollow Nanospheres:

First, 8 mL of TEOS and 280 mL of ethanol were mixed by stirring so as to form a mixture, and 56 mL of deionized water and 8.4 mL of ammonium hydroxide were added thereto, followed by stirring at room temperature for 24 hours and then centrifugation at a speed of 10000 rpm for 10 minutes, so as to form a supernatant and a pellet. Afterward, the supernatant was removed, and the pellet was suspended in 99% of ethanol so as to obtain a suspension. Next, the suspension was subjected to a drying treatment for 6 hours in an oven with a temperature set at 65° C., thereby obtaining silica nanoparticles (abbreviated as silica-NP hereinafter).

Subsequently, 300 mg of the silica-NP was added to 45 mL of ethylene glycol, followed by conducting an ultra-sonication treatment for 30 minutes to avoid the aggregation of the silica-NP, thereby obtaining a silica dispersion. After that, 2.25 mL of 1 M cerium nitrate was added to the silica dispersion, followed by stirring for 10 minutes so as to obtain a mixture. Afterward, the mixture was subjected to a heat treatment for 6 hours using an autoclave with a temperature set at 130° C. to obtain a heated mixture, thereby forming silica-ceria core-shell nanoparticles in the heated mixture. Next, the heated mixture was cooled down to room temperature, followed by centrifugation at a speed of 22000 rpm for 10 minutes, so as to form supernatant and pellet fractions. The supernatant was then removed, and the pellet was washed with 99% of ethanol, thereby obtaining the silica-ceria core-shell nanoparticles (abbreviated as SiO₂—CeO₂-NP hereinafter).

Afterward, the SiO₂—CeO₂-NP thus obtained was subjected to an etching treatment for 48 hours using an 8 N of sodium hydroxide solution serving as an etchant. On the 24^{th} hour after starting the etching treatment, the etchant was replaced with a fresh one so as to remove the silica core. After centrifugation at a speed of 10000 rpm for 10 minutes, the resultant pellet was collected and then suspended in 99% of ethanol, followed by air drying, thereby obtaining ceria hollow nanospheres (abbreviated as CeO₂-HNS hereinafter) having negative charge.

### B. Functionalization of CeO₂-HNS:

The CeO₂-HNS was mixed with the PO-PEG-COOH in a weight ratio of 1:10, followed by suspension in deionized water having an equal volume to a total volume of the CeO₂-HNS and the PO-PEG-COOH, so as to form a mixture. Next, the pH value of the mixture was adjusted to 3 using 1 N solution of hydrochloric acid, followed by ultrafiltration with an ultrafiltration disc (Milipore, 30 kDa NMW, Cat. No. PLTK07610) to remove the liquid portion, thereby obtaining PEG-modified CeO₂-HNS.

After that, 1 mg of the PEG-modified CeO₂-HNS was suspended in deionized water, followed by adding to 5 mL of 2-(N-morpholino)ethanesulfonic acid (MES) buffer containing 0.26 mg of EDC and then stirring for 6 hours. The foregoing process was repeated in triplicates so as to obtain three mixing solutions. After that, the three mixing solutions were added respectively with 5 mL of MES buffer containing 0.25 mg of poly-L-histidine, 5 mL of MES buffer containing 0.5 mg of poly-L-histidine, and 5 mL of MES buffer containing 1 mg of poly-L-histidine, thereby obtaining mixtures 1 to 3. The amounts of the PEG-modified CeO₂-HNS and poly-L-histidine contained in each of the mixtures 1 to 3 were shown in Table 1 below. Each of the mixtures 1 to 3 was subjected to a grafting reaction for 24 hours at room temperature, and then was left at 50° C. for precipitation, so as to form a supernatant and a precipitate, followed by removing the supernatant containing the non-bonded poly-L-histidine. After that, the precipitated was collected and washed with deionized water three times, followed by suspension in deionized water and centrifugation at a speed of 22000 rpm for 10 minutes so as to form supernatant and pellet fractions. Next, the supernatant was removed, and the resultant pellet, i.e., poly-L-histidine functionalized ceria hollow nanospheres (abbreviated as His-CeO₂-HNS), was collected. Finally, the His-CeO₂-HNS were subjected to lyophilization at −50° C. for 24 hours, thereby obtaining a lyophilized powder of His-CeO₂-HNS1, a lyophilized powder of His-CeO₂-HNS2, and a lyophilized powder of His-CeO₂-HNS3.

TABLE 1

| | Lyophilized | Amount (mg) | |
|---|---|---|---|
| Mixture | powder | CeO₂—HNS | Poly-L-histidine |
| 1 | His-CeO₂—HNS1 | 1 | 0.25 |
| 2 | His-CeO₂—HNS2 | 1 | 0.5 |
| 3 | His-CeO₂—HNS3 | 1 | 1 |

In addition, for comparison purposes, poly-L-histidine functionalized ceria nanoparticles (abbreviated as His-CeO₂-NP) was prepared using a method slightly modified from that described by Dai S. et al. (2018), New J. Chem., 42, 18159-18165.

### Example 2. Property Evaluation of Ceria Hollow Nanospheres

### A. Fourier Transform Infrared Spectroscopy (FTIR) Analysis

A suitable amount of a respective one of CeO₂-HNS, and His-CeO₂-HN1 to His-CeO₂-HNS3 obtained in Example 1, and poly-L-histidine was subjected to FTIR analysis using an FTIR spectrometer (FT-730) (Horiba, Japan) in attenuated total reflection (ATR) mode, and FTIR spectra were collected over a wavenumber ranging from 4000 cm⁻¹ to 600 cm⁻¹ at a resolution of 8 cm⁻¹.

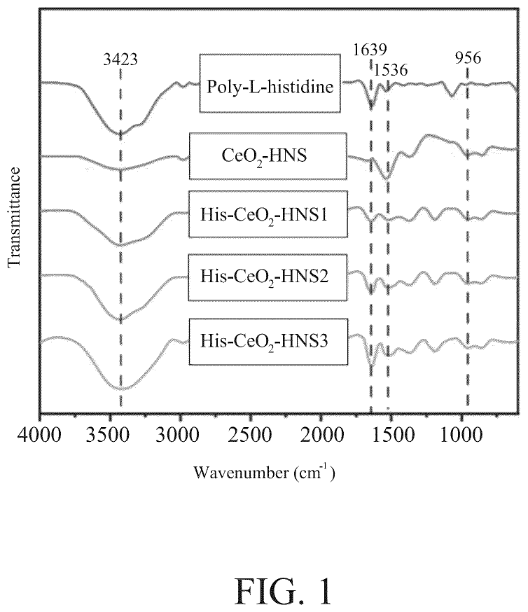

Referring to FIG. 1, the respective one of His-CeO₂-HNS1 to His-CeO₂-HNS3 and poly-L-histidine had an absorption band at 3423 cm-1, while in CeO₂-HNS, no absorption band was observed at 3423 cm⁻¹. These results indicate that each of His-CeO₂-HNS1 to His-CeO₂-HNS3 have poly-L-histidine functional groups.

B. Measurement of zeta (ξ) potential

An appropriate amount of each of CeO₂-HNS, and His-CeO₂-HNS1 to His-CeO₂-HNS3 were dissolved in 10 mM of phosphate buffer (PB) (pH 7.4) to obtain four test samples. Afterward, each of the test samples was subjected to ξ potential measurement using a Zetasizer Nano ZS analyzer (Malvern Instruments, Worcestershire, UK).

The data thus obtained were analyzed according to the method as described in Section 1 of General Procedures.

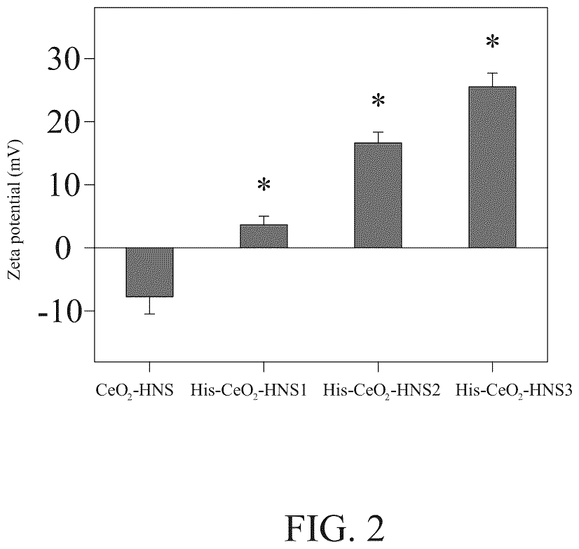

Referring to FIG. 2, CeO₂-HNS had a negative (potential, while each of His-CeO₂-HNS1 to His-CeO₂-HNS3 had a positive (potential. These results indicate that the charge on the surface of each of His-CeO₂-HNS1 to His-CeO₂-HNS3 can be altered by virtue of the functionalization of such ceria hollow nanospheres using poly-L-histidine.

### C. Determination of Particle Size

A suitable amount of a respective one of CeO₂-HNS, His-CeO₂-HNS1 to His-CeO₂-HNS3, and His-CeO₂-NP was subjected to dynamic light scattering (DLS) using the Zetasizer Nano ZS analyzer (Malvern Instruments, Worcestershire, UK), followed by measurement of particle size thereof.

The results show that the each of CeO₂-HNS and His-CeO₂-HNS1 to His-CeO₂-HNS3 had a particle size ranging from 40 nm to 100 nm (average particle size was approximately 75 nm), while His-CeO₂-NP had a particle size ranging from 5 nm to 20 nm. These results indicate that the particle size of ceria hollow nanosphere, i.e., CeO₂-HNS, and His-CeO₂-HNS1 to His-CeO₂-HNS3, is significantly greater than that of ceria nanoparticles, i.e., His-CeO₂-NP.

### D. Morphological Observation

The morphology of each of CeO₂-HNS, His-CeO₂-HNS1 to His-CeO₂-HNS3, and His-CeO₂-NP was observed and photographed using a transmission electron microscope (TEM) (JEOL, Ltd., Model: JSM-1200EX 11).

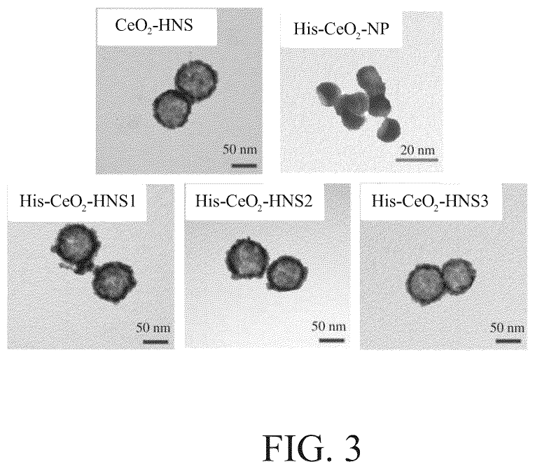

Referring to FIG. 3, each of His-CeO₂-HNS1 to His-CeO₂-HNS3, and CeO₂-HNS had a hollow structure, while in His-CeO₂-NP, no hollow structure was observed.

### E. Determination of Permeability Coefficient

First, an appropriate amount of each of CeO₂-HNS, His-CeO₂-HNS1 to His-CeO₂-HNS3, and His-CeO₂-NP was dissolved in phosphate-buffered saline (PBS), so as to obtain a test solution having a concentration of 1 mg/mL.

Next, the rabbit corneal epithelial cells (RCECs) obtained in Section 5 of General Experimental Materials were divided into six groups, namely, a normal control group, two comparative groups (i.e., comparative groups 1 to 2), and three experimental groups (i.e., experimental groups 1 to 3) (n=2 per group). Each group of the RCECs was seeded at 1×10⁵ cells/well in Transwell inserts (with a polycarbonate membrane having 0.4 μM pores) (Corning Inc.), each of which contained 0.6 mL of the DMEM/F12. The Transwell inserts were then transferred into a respective well of 6-well culture plates containing 1 mL of the DMEM/F12 in each well, followed by cultivation for 24 hours in an incubator with culture conditions set at 37° C., 5% CO₂. Afterward, the RCECs in the comparative group 1 were treated with 1 mL of the CeO₂-HNS test solution, and the RCECs in the comparative group 2 were treated with 1 mL of the His-CeO₂-NP test solution. In addition, the RCECs in the experimental groups 1 to 3 were treated with 1 mL of the His-CeO₂-HNS1 test solution, 1 mL of the His-CeO₂-HNS2 test solution, and 1 mL of the His-CeO₂-HNS3 test solution, respectively. The RCECs in the normal control group received no treatment (with only culture medium). Subsequently, the Transwell inserts of each group were transferred into a respective well of other 6-well culture plates seeded with the rabbit corneal keratocytes (RCKs) and containing 1 mL of the DMEM/F12 in each well, followed by co-cultivation in an incubator (37° C., 5% CO₂) for 0.5 hours and 4 hours, respectively.

On the 0.5^{th} and 4^{th} hour after starting co-cultivation, the Transwell inserts of each group was taken out. The liquid in the respective well of the 6-well culture plate was removed, and the RCKs therein were then washed with PBS, followed by mixing with 500 μL of PBS so as to form a mixture. Afterward, 50 μL of the mixture was subjected to inductively coupled plasma-optical emission (ICP-OES) analysis using an ICP-OES spectrometer (Agilent, Model: Varian-710-ES) so as to determine a concentration of the ceria hollow nanospheres, thereby obtaining a total amount of the ceria hollow nanospheres. Next, the RCKs were harvested and subjected to 4′,6-diamidino-2-phenylindole (DAPI) staining according to techniques well known to those skilled in the art. The number of the stained cells was counted under a confocal laser scanning microscope (CLSM) (Leica, Heidelberg, Germany) at 40× magnification, followed by analysis using ImageJ Imaging Software to obtain an area stained with DAPI.

Subsequently, the permeability coefficient of the RCECs in each group was calculated using the following Equation (1):

$$A = ( B / C ) / ( D \times E ) \qquad ( 1 )$$

$$where$$

$$A = permeability coefficient ( cm / s )$$

$$B = total amount of the ceria hollow nanospheres ( mg )$$

$$C = time period of co - cultivation ( s )$$

$$D = area stained with D A P I ( {cm}^{2} )$$

$$E = initial density of test solution or$$

$$culture medium ( approximately 1 mg / {cm}^{3} )$$

The data thus obtained were analyzed according to the procedures as described in Section 1 of General Procedures.

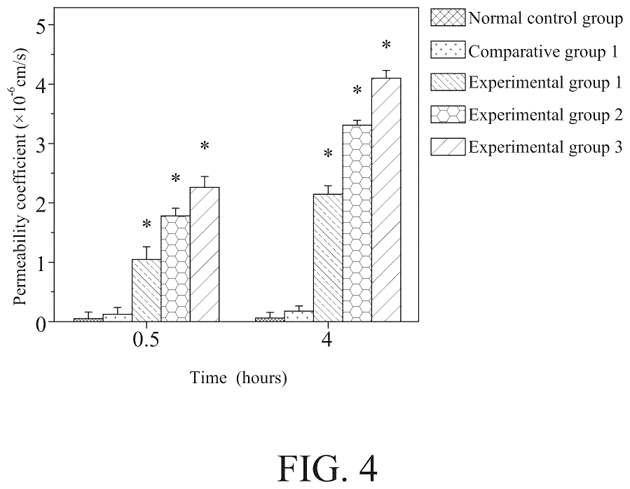

Referring to FIG. 4, the permeability coefficient of the RCECs determined in each of the experimental groups 1 to 3 was significantly greater than those determined in the normal control group and the comparative group 1. In particular, on the 4^{th} hour after co-cultivation, the permeability coefficient of the RCECs determined in each of the experimental groups 1 to 3 surpassed 2×10−6 cm/s while the permeability coefficient of the RCECs determined in each of the normal control group and the comparative group 1 was still lower than 0.5×10⁻⁶ cm/s. These results indicates that His-CeO₂-HNS1 to His-CeO₂-HNS3, each having surface grafted with poly-L-histidine, have excellent permeability in corneal cells.

Moreover, the permeability coefficient of the RCECs determined in the comparative group 2 was lower than 0.2×10⁻⁶ cm/s (data not shown), and no significant difference was observed on the permeability coefficient of the RCECs among the comparative group 2 and the normal control group, indicating that His-CeO₂-NP has little permeability in corneal cells.

### Example 3. Determination of Drug Encapsulation Property of the Ceria Hollow Nanospheres According the Present Disclosure

In this example, the applicants used Ach and SB-431542 encapsulated in the ceria hollow nanospheres according to the present disclosure so as to determine the drug encapsulation property thereof.

First, a suitable amount of each of the CeO₂-HNS test solution, the His-CeO₂-HNS1 test solution, the His-CeO₂-HNS2 test solution, and the His-CeO₂-HNS3 test solution obtained in Section E of Example 2 was mixed with 0.1 μM of Ach solution in a weight ratio of 1:0.6, followed by adding into a vial containing 1.5 mL of balanced salt solution (BSS), so as to form a mixture in which an appropriate amount of hydrogen chloride (HCl) solution might be added in order to maintain the pH value at 6. Next, the mixture was subjected to sonication for 2 hours, and then stirred at room temperature for 24 hours, followed by adjusting the pH value to 7.4 using sodium hydroxide (NaOH) solution under continuous stirring for 12 hours. After that, the stirred mixture was subjected to centrifugation at 15000 rpm for 10 minutes so as to form a precipitation and a supernatant.

Afterward, the supernatant was taken out and then subjected to high performance liquid chromatography (HPLC) analysis using high performance liquid chromatography system (Hitachi, Tokyo, Japan), and an ultraviolet (UV) detector (1L2400, Hitachi), so as to determine an amount of Ach that was not encapsulated by the ceria hollow nanospheres. The operation parameters and conditions for performing HPLC were shown in Table 2 below.

TABLE 2

| | |
|---|---|
| Analytical column | Mighty RP-18 |
| | (Kanto Chemical) |
| Column size | 250 mm × 4.6 mm |
| Measuring wavelength | UV-216 nm |
| Mobile phase | 5% of potassium dihydrogen |
| | phosphate/methanol (85:15, v/v) |
| Flow rate | 0.7 (mL/min) |

Moreover, for comparison purpose, different concentrations of Ach solutions (ranging from 10⁻³ μM to 10 μM), which served as control standards, were prepared and then subjected to the aforesaid HPLC analysis.

Afterward, the amount of Ach encapsulated in a respective kind of the ceria hollow nanospheres (i.e., CeO₂-HNS, and His-CeO₂-HNS1 to His-CeO₂-HNS3) was calculated using the following Equation (2):

$$E = F - G \qquad ( 2 )$$

$$where$$

$$E = amount of Ach encapsulated in ceria hollow nanospheres ( g )$$

$$F = initial amount of Ach ( g )$$

$$G = amount of Ach not encapsulated in ceria hollow nanospheres ( g )$$

The encapsulation percentage of Ach for the respective kind of the ceria hollow nanospheres (i.e., CeO₂-HNS, and His-CeO₂-HNS1 to His-CeO₂-HNS3) was calculated using the following Equation (3):

$$H = ( E / I ) \times 100 \qquad ( 3 )$$

$$where$$

$$H = encapsulation percentage of Ach for ceria hollow nanospheres ( \% )$$

$$E = amount of Ach encapsulated in ceria hollow nanospheres ( g )$$

$$I = amount of ceria hollow nanospheres used ( g )$$

In addition, the encapsulation percentage of SB-431542 of the respective kind of the ceria hollow nanospheres (i.e., CeO₂-HNS, and His-CeO₂-HNS1 to His-CeO₂-HNS3) was determined using procedures similar to those of the Ach as described above, except that 0.1 μM of Ach solution was replaced with 260 μM of SB-431542, and different concentrations of SB-431542 (ranging from 50 μM to 103 μM), which served as control standards, were subjected to the aforesaid HPLC analysis.

The data thus obtained were analyzed according to the procedures as described in Section 1 of General Procedures.

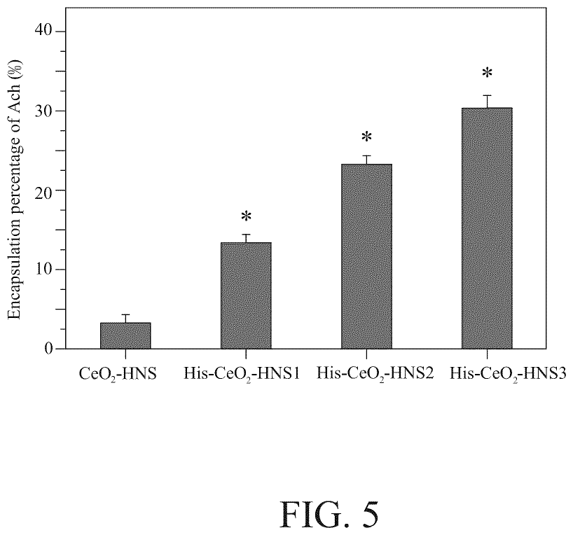

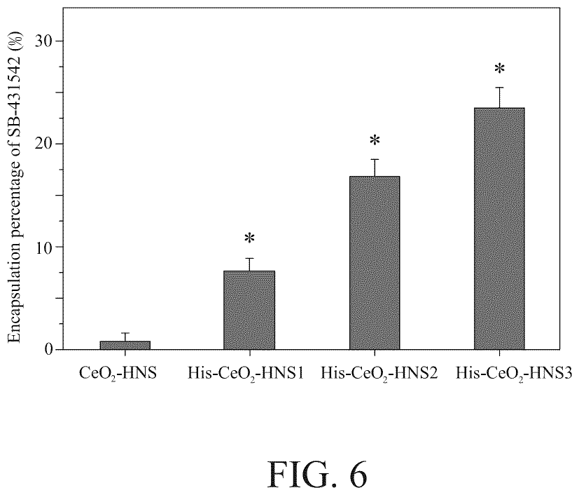

Referring to FIGS. 5 and 6, the encapsulation percentage of the drugs (i.e, Ach or SB-431542) for the respective one of His-CeO₂-HNS1 to His-CeO₂-HNS3 was significantly higher than that of CeO₂-HNS, and among such nanospheres, the encapsulation percentage of the drugs (i.e, Ach or SB-431542) for His-CeO₂-HNS3 was the highest. These results indicate that the ceria hollow nanospheres according to the present disclosure (i.e., His-CeO₂-HNS1 to His-CeO₂-HNS3), in particular His-CeO₂-HNS3, which was obtained by mixing CeO₂-HNS and poly-L-histidine in the weight ratio of 1:1, can effectively encapsulate ophthalmic drugs.

The encapsulation ratio of each drug (i.e, Ach or SB-431542) for the respective kind of ceria hollow nanospheres (i.e., CeO₂-HNS, and His-CeO₂-HNS1 to His-CeO₂-HNS3) was calculated using the following Equation (4):

$$J = [ E / ( 1 + F ) ] \times 100 \qquad ( 4 )$$

$$where$$

$$J = encapsulating ratio of Ach or SB - 431542 ( \% )$$

$$E = amount of Ach or$$

$$SB - 431542 encapsulated in ceria hollow nanospheres ( g )$$

$$I = amount of ceria hollow nanospheres used ( g )$$

$$F = initial amount of Ach or SB - 431542 ( g )$$

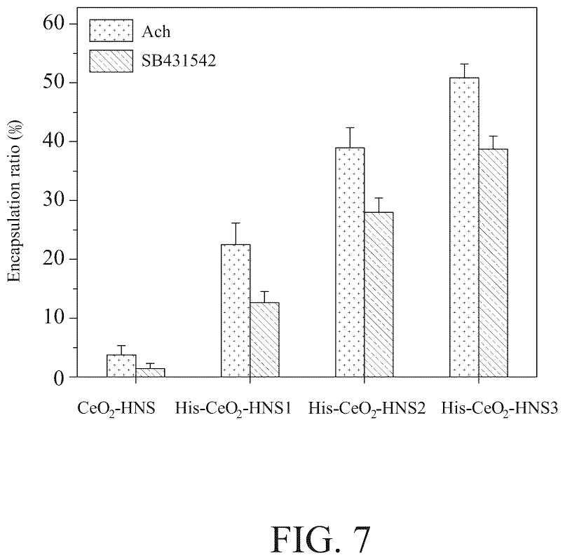

Referring to FIG. 7, the encapsulation ratio of Ach or SB-431542 for the respective one of His-CeO₂-HNS1 to His-CeO₂-HNS3 was significantly greater than that for CeO₂-HNS, and among such nanospheres, the encapsulating ratio of Ach or SB-431542 for His-CeO₂-HNS3 was the greatest. These results indicate that the ceria hollow nanospheres (i.e., His-CeO₂-HNS1 to His-CeO₂-HNS3) according to the present disclosure, in particular His-CeO₂-HNS3, which was obtained by mixing CeO₂-HNS and poly-L-histidine in the weight ratio of 1:1, can effectively encapsulate ophthalmic drugs.

### Example 4. Evaluation of Drug Delivery to Target Site for the Ceria Hollow Nanospheres According to the Present Disclosure

### Experimental Materials:

### 1. Preparation of Test Solution

For comparison purpose, the dual functionalized ceria hollow nanospheres (abbreviated as DF-CeO₂-HNS), which were grafted with both ZM-241385 and chitosan as described in Taiwanese Invention Patent Publication NO. 1764564, was dissolved with PBS so as to obtain a test solution having a concentration of 1 mg/mL.

### Experimental Procedures:

The male SD rats were randomly divided into two groups (n=10 per group), namely, an experimental group and a comparative group, followed by subjecting the SD rats in each group to anesthesia with intramuscular injection of Zoletil (Virbac, Carros, France) (2.5 mg/kg body weight) and xylazine hydrochloride (1 mg/kg body weight). The ocular surface of each of the SD rats in the experimental group was subjected to instillation with 50 μL of the His-CeO₂-HNS3 test solution obtained in Section E of Example 2. In addition, the ocular surface of each of the SD rats in the comparative group was subjected to instillation with 20 μL of the DF-CeO₂-HNS test solution obtained in the section Experimental Materials of this example.

On the 4^{th} day after instillation, the rats in each group were sacrificed using CO₂. After that, approximately 0.5 mg of the corneal tissue was cut from the eyes of each rat in each group using a scalpel blade and then ground with liquid nitrogen, followed by adding 600 μL of RIPA lysis buffer (Santa Cruz, Cat. No. sc-24948) and mixing, so as to obtain a mixture. Next, the mixture was dissolved in deionized water to obtain a test sample having a concentration of 1 mg/mL. Additionally, the concentration of cerium (Ce) in the test samples of each group was determined using the ICP-OES (Agilent, Model: Varian-710-ES), and the relative content of Ce in the test samples of each group was calculated by using following formula Equation (5):

$$K = L / M \qquad ( 5 )$$

$$where$$

$$K = relative content of Ce$$

$$L = concentration of Ce in each group$$

$$M = concentration of Ce in comparative group$$

### Results:

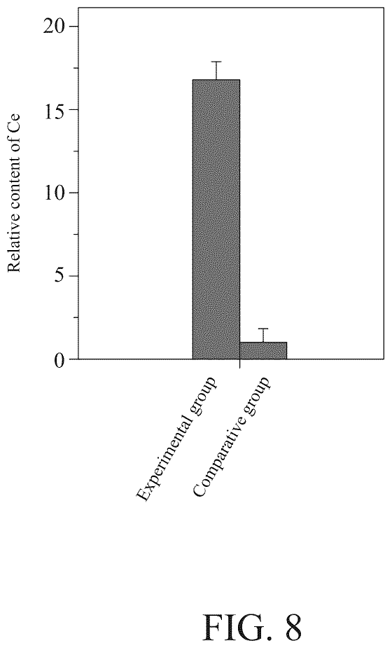

Referring to FIG. 8, Ce could be detected in the corneal tissues of the rats in both the experimental group and the comparative group, and the relative content of Ce in the corneal tissues of the rats in the experimental group was significantly greater than that in the comparative group. These results indicate that the ceria hollow nanospheres (i.e., His-CeO₂-HNS3) according to the present disclosure exhibit an excellent effect in delivering drugs to corneal tissues.

### Example 5. Evaluation of Therapeutic Effect of Ophthalmic Drugs Encapsulated in Ceria Hollow Nanospheres According to the Present Disclosure on Structural Damage of Cornea Caused by Corneal Alkali Burn

### Experimental Materials:

### 1. Preparation of a Solution Containing Ach and SB-431542

Appropriate amounts of Ach and SB-431542 were mixed in a weight ratio of 1:1, so as to obtain a solution containing Ach and SB-431542.

2. Preparation of his-CeO₂-HNS3 Encapsulating Ach and SB-431642

An appropriate amount of the His-CeO₂-HNS3 test solution obtained in Section E of Experimental 2 and an appropriate amount of the solution containing Ach and SB-431542 were mixed in a weight ratio of 1:1, followed by adding into a vial containing 1.5 mL of BBS and adjusting the pH value to 6 using HCl solution (if required), so as to form a mixture. Next, the mixture was subjected to sonication for 2 hours, and then stirred at room temperature for 24 hours, followed by adjusting the pH value to 7.4 using NaOH solution and continuously stirring for 12 hours. Subsequently, the stirred mixture was subjected to centrifugation at 15000 rpm for 10 minutes so as to form a precipitation and a supernatant. After that, the supernatant was removed, and then the precipitate was washed with PBS three times, thereby obtaining the His-CeO₂-HNS3 encapsulating Ach and SB-431542.

### Experimental Procedures and Results:

### A. Induction of Corneal Alkali Burn:

A round filter paper having a diameter of approximately 4 mm was soaked in 1 N of NaOH solution, followed by placing the NaOH-soaked filter paper on a central corneal surface of an SD rat for 30 seconds, and then the washing the central corneal surface with 30 mL of sterile saline water, so as to induce corneal alkali burn.

### B. Administration of Ach and SB-431542:

30 male SD rats were subjected to the induction of corneal alkali burn using the method as described in Section A above, and then were randomly divided into two groups (n=10 per group), namely, a pathological control group and an experimental group. After that, the rats in each group were subjected to anesthesia according to the method described in Experimental Procedures of Example 4. Next, each rat in the experimental group was subjected to ocular topical instillation using the His-CeO₂-HNS3 encapsulating Ach and SB-431542 (20 μL per rat). The rats in the pathological control group received no treatment.

Moreover, the male SD rats which were not subjected to induction of corneal alkali burn and ocular topical instillation using the His-CeO₂-HNS3 encapsulating Ach and SB-431542, was named as the normal control group (n=10).

### C. Evaluation of Corneal Haziness:

On the 4^{th} day after administration of Ach and SB-431542, the rats in each group were sacrificed using CO₂. After that, the corneal tissue was cut from the eyes of each rat using a scalpel blade, and then the cut corneal tissue was placed on a typescript marked with the letter “A”, followed by observing and photographing the corneal tissue using a slit lamp biomicroscope (Topcon Optical).

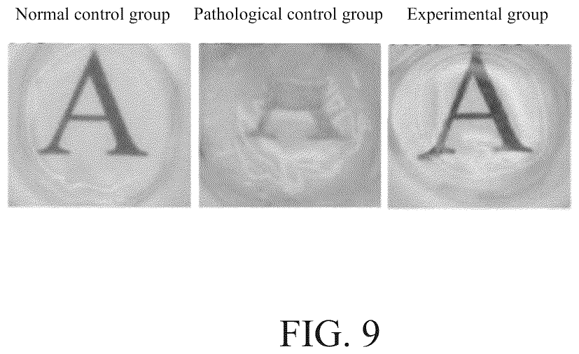

Referring to FIG. 9, the corneal haziness of the corneal tissue in pathological control group was significantly greater compared with that of the normal control group, indicating that the induction of corneal alkali burn decreases the transparency of a cornea. In comparison with the pathological control group, the corneal haziness of the corneal tissue in the experimental group was significantly less than and was even close to that of the corneal tissue in the normal control group. These results indicate that His-CeO₂-HNS3 having surface grafted with poly-L-histidine can not only effectively encapsulate ophthalmic drugs, but also deliver the ophthalmic drugs to the corneal tissue, thereby achieving the effect of alleviating corneal haziness caused by corneal alkali burn.

### D. Histological Analysis:

First, 50 mg of the corneal tissues of the rats in each group which were obtained in Section C above to serve as a histological sample, was subjected to fixation with 4% paraformaldehyde (in PBS) at room temperature for 30 minutes, followed by embedding with paraffin and slicing, so as to obtain a tissue section having a thickness of 5 μm.

Afterward, the tissue section was subjected to hematoxylin-eosin staining using a staining protocol well-known to those skilled in the art. A region of the tissue section, which was randomly selected, was subjected to photography and histological observation using an optical microscope (Nikon, Tokyo, Japan) at a magnification of 20×.

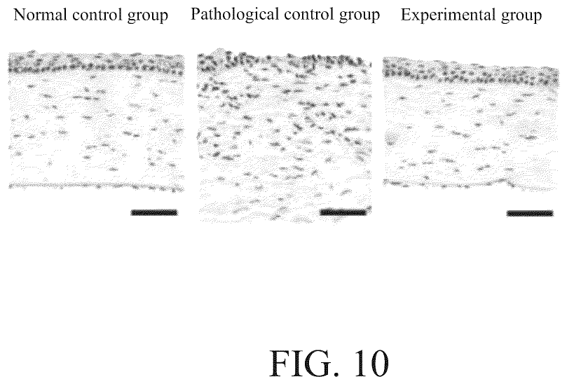

Referring to FIG. 10, the corneal tissue of the rats in the pathological control group showed a multi-layered tissue microstructure having high degree of integrity destruction and swollen stromal layers, indicating that corneal alkali burn damages the structure of the corneal tissue. In contrast, the corneal tissue in the respective one of the normal control group and the experimental group did not show such multi-layered tissue microstructure. These results indicate that His-CeO₂-HNS3 having surface grafted with poly-L-histidine can not only effectively encapsulate ophthalmic drugs, but also deliver the ophthalmic drugs to the corneal tissue, thereby achieving the effect of alleviating the structural damage of a cornea caused by corneal alkali burn.

### E. Evaluation of Young's Modulus:

The evaluation of Young's modulus of the tissue section of each group obtained in Section D was conducted using an universal testing machine (Instron, Canton, MA, USA) at a crosshead speed of 0.5 mm/min. The higher the Young's coefficient is, the better the tension of the tissue section is.

The data thus obtained were analyzed according to the procedures as described in Section 1 of General Procedures.

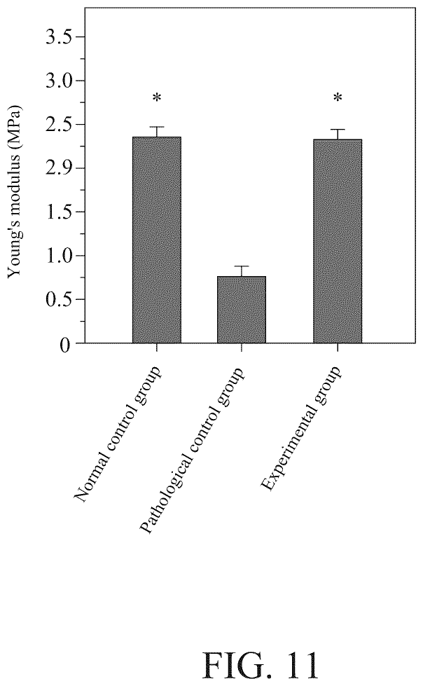

Referring to FIG. 11, the Young's coefficient determined in the pathological control group was significantly less than that of the normal control group, indicating that the tension of the corneal tissue decreased due to corneal alkali burn. In addition, the Young's coefficient determined in the experimental group was significantly greater or even was close to that of the normal control group. These results indicate that His-CeO₂-HNS3 having surface grafted with poly-L-histidine can not only effectively encapsulate ophthalmic drugs, but also deliver the ophthalmic drugs to the corneal tissue, thereby alleviating tension damage of the corneal tissue caused by corneal alkali burn.

### Example 6. Evaluation of Therapeutic Effect of Ophthalmic Drugs Encapsulated in Ceria Hollow Nanospheres According to the Present Disclosure on the Corneal Inflammatory Damage Caused by Corneal Alkali Burn

### Experimental Materials:

1. Preparation of his-CeO₂-HNS Carrying Dexamethasone:

The procedures for preparing His-CeO₂-HNS encapsulating dexamethasone were similar to those described in Section 2 of the Experimental Materials in Example 5, except that Ach and SB-431542 were replaced with dexamethasone.

### Experimental Procedures and Results:

### A. Induction of Corneal Alkali Burn and Administration of an Ophthalmic Drug:

First, 30 male SD rats were subjected to induction of corneal alkali burn according to the method as described in Section A of Example 5, followed by randomly dividing the rats into 3 groups (n=10 per group), namely, a pathological control group and two experimental groups (i.e., experimental groups 1 and 2). Next, each rat in the experimental group 1 was subjected to ocular topical instillation using the His-CeO₂-HNS3 encapsulating dexamethasone (20 μL per rat) obtained in Section 1 of Experimental Materials. In addition, each rat in the experimental group 2 was subjected to ocular topical instillation using the His-CeO₂-HNS3 encapsulating Ach and SB-431542 (20 μL per rat). The rats in the pathological control group received no treatment.

Moreover, male SD rats which were not subjected to induction of corneal alkali burn and ocular topical instillation using the His-CeO₂-HNS3 encapsulating dexamethasone, was named as the normal control group.

B. Determination of Relative mRNA Expression Level of IL-13 Gene

On the 4^{th} day after ocular topical instillation, the rats in each group were sacrificed using CO₂. After that, 50 mg of the corneal tissues were cut from the eyes of the rats in each group using a scalpel blade, and then was subjected to total RNA extraction using Qiagen RNeasy Mini Kit (Qiagen, Hilden, Germany) in accordance with the manufacturer's instructions, so as to obtain total RNA. The total RNA was then subjected to reverse transcription reaction using a reverse transcription reagent (Promega Reverse Transcriptase, Cat. No. WI 53711-5399) in accordance with the manufacturer's instructions, so as to synthesize a first-strand complementary DNA (cDNA).

Subsequently, the first-strand cDNA was used as a template and subjected to quantitative real-time polymerase chain reaction (qRT-PCR) with specific primer pair designed for IL-1β gene, with reference to Lai J. Y. et al. (2014), Carbohydrate Polymers, 101:203-212. Additionally, glyceraldehyde-3-phosphate dehydrogenase (GAPDH) gene was expressed to be used as internal control. The information regarding the primer pairs (e.g., target genes, nucleotide sequences of each primer pair, and sizes of PCR products) was shown in Table 3 below.

TABLE 3

| | | | Size of |
|---|---|---|---|
| | | Nucleotide | PCR |
| | Primer | sequence | product |
| Target gene | pair | (5′ → 3′) | (bp) |
| IL-1β | Forward | gcaactgttcc | 307 |
| (corresponding | prime | tgaactcaact | |
| to GenBank | (IL-1β- | (SEQ ID | |
| Accession | F) | NO: 1) | |
| Number | | | |
| NM_008361) | | | |
| | Reverse | atcttttgggg | |
| | primer | tccgtcaact | |
| | (IL-1β- | (SEQ ID | |
| | R) | NO: 2) | |
| GAPDH | Forward | tggtatcgtg | 189 |
| (corresponding | primer | gaaggactca | |
| to GenBank | (GAPDH- | tgac | |
| Accession | F) | (SEQ ID | |
| | | NO: 3) | |
| Number | Reverse | atgccagtgag | |
| XM_ | primer | cttcccgttca | |
| 001726954.1) | (GAPDH- | gc | |
| | R) | (SEQ ID | |
| | | NO: 4) | |

The qRT-PCR was carried out using ABI 7900HT Real Time PCR system (Applied Biosystems) in accordance with the manufacturer's instructions sp as to obtain PCR products, and the procedures and conditions of the qRT-PCR as shown in Table 4 below, were generally based on the method described in Ma C. et al. (2015), PLoS One., 10(5):e0125776.

TABLE 4

| Contents | Volume (μL) |
|---|---|
| First-strand cDNA (0.1 μg/μL) | 1 |
| Forward primer (10 μM) | 0.8 |
| Reverser primer (10 μM) | 0.8 |
| Maxima SYBR ® Green/Fluorescein qPCR Master | 5 |
| Mix (2X) (Applied Biosystems, Cat. No. 4309155) | |
| Deionized water | 3.4 |

Operation conditions: 40 cycles of the following reaction: denaturation at
95° C. for 5 minutes, primer annealing at 95° C. for 10 seconds, and
extension at 60° C. for 30 seconds.

The PCR products thus obtained were subjected to SYBR® Green (a double-stranded DNA dye) fluorescence detection so as to determine the cycle threshold (Ct) value of IL-1β gene in each group. The relative mRNA expression level of IL-1β gene in each group was obtained using the comparative Ct method in which the Ct value of IL-1β gene was normalized with the Ct value of GAPDH gene, followed by subtracting the normalized Ct value of IL-1β gene in the normal control group.

The data thus obtained were analyzed according to the procedures as described in Section 1 of General Procedures.

### Results:

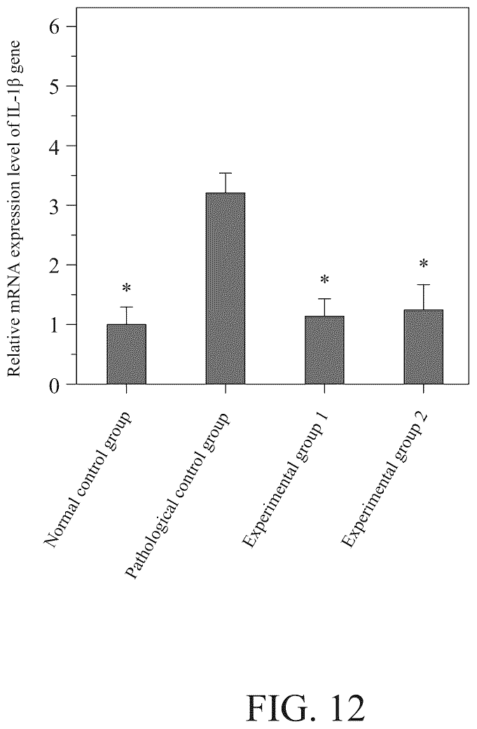

Referring to FIG. 12, in comparison with the normal control group, the corneal tissues of the rats in the pathological control group had increased expression of IL-1β gene, indicating that corneal alkali burn induced inflammatory response in the corneal tissues. In addition, in comparison to the pathological control group, the relative mRNA expression level of IL-1β gene in each of the experimental groups 1 and 2 was significantly decreased or even was close to that of the normal control group. These results indicate that His-CeO₂-HNS3 having surface grafted with poly-L-histidine can not only effectively encapsulate ophthalmic drugs, but also deliver the ophthalmic drugs to the corneal tissues, thereby achieving the effect of alleviating the inflammatory damage caused by corneal alkali burn.

Summarizing the above results, through functionalization using poly-L-histidine, the ceria hollow nanospheres can be conferred with an excellent corneal permeability, so as to effectively deliver ophthalmic drugs encapsulated therein to the anterior eye segment tissues and to rapidly release such ophthalmic drugs, thereby achieving the effect of alleviating an anterior segment eye disease.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A nanosphere, comprising:
a metal oxide hollow nanosphere, the metal oxide being selected from the group consisting of cerium(IV) oxide (CeO₂), aluminum oxide (Al₂O₃), copper(II) oxide (CuO), titanium dioxide (TiO₂), sodium oxide (Na₂O), zinc oxide (ZnO), gold(II) oxide (AuO), iron(II, III) oxide (Fe₃O₄), and combinations thereof; and
a poly-L-histidine grafted on a surface of the metal oxide hollow nanosphere.

2. The nanosphere as claimed in claim 1, wherein the metal oxide is CeO₂.

3. The nanosphere as claimed in claim 1, which is produced by subjecting the metal oxide hollow nanosphere and the poly-L-histidine to a grafting reaction, wherein a weight ratio of the metal oxide hollow nanosphere to the poly-L-histidine ranges from 1:0.25 to 1:1.

4. The nanosphere as claimed in claim 3, wherein before the grafting reaction, the surface of the metal oxide hollow nanosphere is subjected to modification using polyethylene glycol.

5. The nanosphere as claimed in claim 1, wherein the metal oxide hollow nanosphere has a particle size ranging from 20 nm to 150 nm.

6. A drug delivery carrier, comprising:
a nanosphere as claimed in claim 1; and
an ophthalmic drug.

7. The drug delivery carrier as claimed in claim 6, wherein a weight ratio of the nanosphere to the ophthalmic drug ranges from 1:0.5 to 1:10.

8. The drug delivery carrier as claimed in claim 6, wherein the ophthalmic drug is selected from the group consisting of acetylcholine chloride, 4-[4-(1,3-benzodioxol-5-yl)-5-(2-pyridinyl)-1H-imidazol-2-yl]benzamide (SB-431542), tobramycin, penicillin, tetracycline, dexamethasone, hydrocortisone, hydrocortisone acetate, amcinonide, betamethasone, halometasone, clobetasone-17-butyrate, ascorbic acid, hyaluronic acid, and combinations thereof.

9. A method for alleviating an anterior segment eye disease, comprising administrating to a subject in need thereof a pharmaceutical composition containing a drug delivery carrier as claimed in claim 6.

10. The method as claimed in claim 9, wherein the anterior segment eye disease is selected from the group consisting of ocular burn, keratitis, uveitis, conjunctivitis, xerophthalmia, endophthalmitis, obstruction of meibomian glands, and combinations thereof.

11. The method as claimed in claim 9, wherein the pharmaceutical composition is in a dosage form for intraocular administration or topical ophthalmic administration.
